(19) United States
(12) Patent Application Publication
(10) Pub. No.: US 2014/0377839 A1
(43) Pub. Date: Dec. 25, 2014

(54) MONOATOMIC POINT MUTANT ARTIFICIAL OXIDOREDUCTASES

(71) Applicant: Aron S. Workman, Gainesville, FL (US)

(72) Inventor: Aron S. Workman, Gainesville, FL (US)

(21) Appl. No.: 13/924,451

(22) Filed: Jun. 21, 2013

(51) Int. Cl. C12N 9/02

(52) U.S. Cl. CPC C12N 9/0089
USPC 435/189

(57) ABSTRACT

This invention in biotechnology/bioinformatics claims artificial mutant polypeptides of an oxidoreductase wherein the wild-type (unmutated) oxidoreductase is human catalase (hcat), a superoxide dismutase isoenzyme of superoxide dismutase 2 (hsod2) or superoxide dismutase 3 (hsod3), or a human glutathione peroxidase isoenzyme of human glutathione peroxidase 1 isoform 1 (hgpx1-1), human glutathione peroxidase 1 isoform 2 (hgpx1-2), human glutathione peroxidase 2 (hgpx2), or human glutathione peroxidase 3 (hgpx3). Disclosed in the specification are the methods by which the monoatomic point mutant libraries have been constructed, and the claimed oxidoreductase polypeptides' encoding nucleic acids and recombinant cells thereof. The monoatomic point mutant method generates artificial polypeptides with altered function whilst limiting primary structural obfuscation. The claimed products have multiple potential industrial applications including as novel therapeutics and industrial catalysts.

### CROSS-REFERENCE TO RELATED APPLICATIONS

Priority is claimed from U.S. provisional application Ser. No. 61/662,605, filed 21 Jun. 2012.

### BACKGROUND OF THE INVENTION

This invention pertains to molecular biology. More specifically, this invention pertains to mutant oxidoreductase polypeptides.

Oxidoreductases are polypeptides that catalyze electron transfer. Electrons are the reactive component of reactive oxygenated species (ROS) and many oxidoreductases are also antioxidants.

Antioxidants are historical therapeutics that attenuate inflammation. Oxidoreductases may also be utilized to catalyze industrial reactions. Thus there is interest in developing oxidoreductases with improved industrial properties.

One method of improving industrial properties of polypeptides is to introduce site-specific mutations. An artificial point mutation is the act of substituting a natural peptide residue for an artificial one.

Some methods by which introduced mutations have been chosen include analogy, homology, understanding of the polypeptide/polypeptide system, or from structure-function studies.

Multitudinous published and issued U.S. patents claim such mutations and methods.

### BRIEF SUMMARY OF THE INVENTION

Oxidoreductases were systematically mutated by generating monoatomic point mutant (MPM) libraries.

A MPM is one in which the resultant residue differs by only a single atom excluding hydrogen atoms.

The Glu/Gln and Asp/Asn MPM pairs were applied to the oxidoreductases hcat; superoxide dismutase isoenzymes hsod2 and hsod3; and glutathione peroxidase isoenzymes hgpx1-1, hgpx1-2, hgpx2, and hgpx3.

### BRIEF DESCRIPTION OF THE DRAWING

FIG. 1 shows the primary residue structures of the defined MPM pairs Glu/Gln and Asp/Asn by stick (cartoon) illustration.

### DETAILED DESCRIPTION OF THE INVENTION

The present invention uses terms of the art including “nucleic acid”, “polynucleotide”, “wild-type”, “recombinant”, “isoenzyme”, “isoform”, “transgenic”, “variant”, “residue”, “mutant”, “mutation”, “substitution”, “oxidoreductase”, “primary”, “primary structure”, and etcetera. These terms are widely used and understood in the fields of biochemistry and molecular biology (Setubal C & Meidanis J (1997) Introduction to Computational Molecular Biology, PWS Publishing, pp 320; Karp G (2007) Cell and Molecular Biology: Concepts and Experiments, Wiley, pp 864; Sambrook J (2001) Molecular Cloning: A Laboratory Manual, Cold Spring Harbor Laboratory Press, pp 999).

“Nucleic acid” or “polynucleotides” refer to either DNA or RNA, or both DNA and RNA. A “biomolecule” refers to both the nucleic acid and polypeptide products of a single molecule definable by its polypeptide sequence according to central dogma; that is, a polypeptide and all its encoding nucleic acids.

While “isoenzyme” and “isoform” are sometimes colloquially interchangeable or ambiguous terms, herein “isoenzymes” refer to polypeptides that catalyze an identical net reaction but with differing sequence generated from differing nucleic acid, whereas “isoform” refers to a polypeptide with an identical net reaction and shared sequence domains from shared nucleic acid. Differing sequences of isoenzymes of course do not necessarily preclude homological or otherwise comparable sequence domains.

“Artificial” refers to a non-naturally occurring polypeptide or nucleic acid; that is, a biomolecule that was not previously a facet of nature before introduction by invented human methods.

There are differing standards of polypeptide nomenclature and residue assignment.

Differing nomenclature occurs throughout the normal process of polypeptide discovery that in no way affects physical polypeptide or nucleic acid sequence.

Differing residue assignments are historical artifacts that vary based on conventions or standards specific to each polypeptide.

One origin of differing residue assignments is that different synthetic systems (both natural or by human invention) may or may not cleave or include the N-terminal methionine. In identifying polypeptide residues, residue number N-1 is residue number N if the N-terminal methionine is included in the sequence representation. This assignment is not to be confused with the physical sequence: many translated biomolecules begin with the start codon N-terminal methionine, but not all sequence assignments explicate this (for example, the N-terminal methionine may, if pertinent, be assigned residue 0). For example, the residue Glu38 is Glu39 if the N-terminal methionine is included. For consistency, sequences here are listed without the N-terminal methionine. That sequence is included in and contained by either standard sequence.

A single atom substitution mutant, or monoatomic point mutant (MPM) (or plural: MPMs) polypeptide is mutation wherein a single atom (or atoms) change per mutant residue excepting hydrogen atoms. For example, Cys to Sec is an MPM because the net mutation is the substitution of a sulfur atom for a selenium atom with all other atoms held identical, and conversely Sec to Cys is selenium to sulfur. Every MPM has a converse, and these two mutations are together called an MPM pair, identified by residue, for example the MPM pair Sec/Cys.

Hydrogen atoms are not included in MPM designations because their presence is largely dependent on the acidity/basicity (solvated hydrogen ion concentrations or pH) of the milieu, and thus vary from polypeptide to polypeptide and from milieu to milieu.

(During the course of discussion on MPM and MPMs polypeptides, the identifier ‘polypeptide’ may be omitted for clarity).

Some other MPM types are: Asp to Asn (oxygen to nitrogen) and its converse Asn to Asp (nitrogen to oxygen); and Glu to Gln (oxygen to nitrogen) and its converse Gln to Glu (nitrogen to oxygen).

While an MPM is a single atom substitution within a single residue, a single polypeptide may contain multiple MPM substitutions at different residues (MPMs). For example, while a polypeptide containing three Cys to Sec MPM residues is a polymutant polypeptide (also called a compound mutant polypeptide), it is also (and more specifically) a MPMs polypeptide.

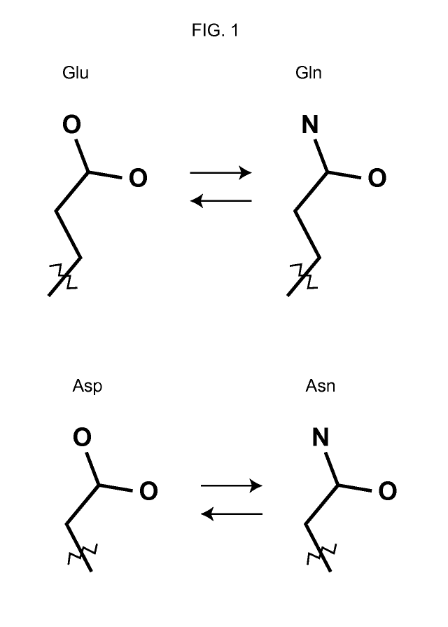

Note the scale on which mutation is discussed. For example, a polymutant may more generally refer to an organism or system with multiple distinct mutant polypeptides, but in the instances disclosed herein clearly refers to multiple mutations within a single polypeptide. Like other chemical descriptions, graphical representations greatly alleviate the lexical burden associated with describing such systems. See FIG. 1 for a graphic of the MPM pairs Glu/Gln and Asp/Asn.

MPM polypeptides are useful because they are as a group the least primary structurally disruptive mutant variants available. Thus MPM libraries, or sets of MPM and MPMs variants of a particular polypeptide, allow screening and analysis of a particular polypeptide's least primary structurally disruptive mutant variants.

Minimizing structural disruption in mutant polypeptide screening is advantageous because polypeptides evolve precise structures that are sensitive to structural disruption. Particular point mutant residues are known to destabilize, inactivate, or otherwise incapacitate particular polypeptides, especially if these mutants initiate large steric or electronic changes in the polypeptide.

For example, the MPM Cys to Sec affects the polypeptide's primary structure much less than a Cys to Pro or Cys to Trp mutation at the same residue, in part because the MPM is a significantly more limited steric and electronic change.

Thus it is posited that an MPM is the most primary incremental point mutation available. This is not obvious because of other similarities between variants that are not MPM. For example, to minimize structural disruption to a Cys residue researchers and technologists often opt for the Cys to Met mutant, a non-MPM, because both residues contain sulfur. The Cys to Met mutant has a net introduction of two carbon atoms excluding hydrogen atoms.

For the MPM pairs claimed, both Glu and Asp contribute negative electric charge to the polypeptide whereas both Gln and Asn are electrically neutral. These MPM pairs allow changing the electrical properties of a polypeptide with minimal structural obfuscation, which often generate advantageous industrial properties.

Each polypeptide claimed pertains to molecular biology. More specifically, each polypeptide claimed is an oxidoreductase polypeptide. Each polypeptide is also entirely artificial: the correspondingly mentioned “wild-type (unmutated)” sequence is simply a reference for organization of sequences.

Human superoxide dismutase isoenzymes (Enzyme Commission number 1.15.1.1) are oxidoreductase polypeptides that are thought to primarily catalyze the reaction: 2 O₂⁻+2 H⁺→O₂+H₂O₂. Superoxide dismutase 2 (hsod2) and superoxide dismutase 3 (hsod3) are examples of human superoxide dismutase isoenzymes.

Human catalase (hcat) (Enzyme Commission number 1.11.1.6) is an oxidoreductase polypeptide that is thought to primarily catalyze the reaction: 2 H₂O₂→2 H₂O+O₂.

Human glutathione peroxidase isoenzymes (Enzyme Commission number 1.11.1.9) are selenium-containing oxidoreductase polypeptides that are thought to primarily catalyze the reaction: 2 glutathione+H₂O₂→glutathione disulfide+2 H₂O. Glutathione peroxidase 1 isoform 1 (hgpx1-1), glutathione peroxidase 1 isoform 2 (hgpx1-2), glutathione peroxidase 2 (hgpx2), and glutathione peroxidase 3 (hgpx3) are examples of human glutathione peroxidase isoenzymes. Except for five N-terminal amino acids, gpx1-2 is entirely contained in gpx1-1.

Human thioredoxin, human superoxide reductase, human myeloperoxidase, and human glutathione reductase as sequence listed are other oxidoreductase polypeptides, both known isoenzymes and without known isoenzymes, that are thought to primarily catalyze oxidoreductive reactions.

It is possible to generate the artificial nucleic acid sequences encoding the artificial polypeptide sequences by consulting published codon tables of the appropriate organism. Due to codon degeneracy, multitudinous artificial nucleic acid sequences—cDNA, plasmid, and otherwise—may encode one polypeptide product. While the encoding nucleic acids and resultant polypeptide are often seen as a single biomolecular entity, when nucleic acid are necessarily claimed they are identified by their resultant polypeptide product. The polypeptide may then be translated back to nucleic acids, or the nucleic acids translated to polypeptide, to discover infringement. This is a trivial translation for one skilled in the art.

Transformation/transfection of encoding nucleic acid of any number of cells of multitudinous hosts is a trivial procedure for one skilled in the art. Resultant recombinant cells are not limited by species, transformation method, expression levels of any kind, or any other qualifier.

Methods of use, synthesis, and purification may or may not vary for each of the claimed products. Generally, the claimed products are being developed as viable recombinant therapeutics and to catalyze industrial (especially radical-mediated) reactions. Oxidoreductase (and antioxidant) therapeutics are, as a historical matter, particularly efficacious for the treatment of inflammation.

Without further elaboration, it is believed that one skilled in the art can, using the disclosed method (or mutant identities), utilize the present invention and its best mode to its fullest extent. The following specific embodiments are therefore merely illustrative and are not limitative of the remainder of the disclosure or claims in any way whatsoever:

### EXAMPLE 1

### Preparation of MPM Library

Wild-type sequence data were obtained and aligned for validity from multiple public sources.

To acquire the MPM library with least human error, a simple Perl script was written and executed from console. The regular expression was changed to generate mutant sequences with start codon M included or from base sequences that included special characters. The script was run for each MPM type. Generation of all possible MPMs is possible by sequential mutation or modified script.

A functional Perl script in its entirety is:

open INPUT, $ARGV[0] or die $!; my @input =
grep(s/\d//g|s/\R//g|s/\s+//g|s/\hat{ }M//|s/\hat{ }m//, <INPUT>); close INPUT; my ($input) = join(“,
@input); $input = uc($input); open WT, “> wt−$ARGV[0]” or die $!; print WT $input;
close WT; print “Target AA: ”; my $char = <STDIN>; chomp $char; $char = uc($char);
exit 0 if ($char eq “”); print “Mutant AA: ”; my $opchar = <STDIN>; chomp $opchar;
$opchar = uc($opchar); exit 0 if ($opchar eq “”); my $length = length($input); print
“Generating $char to $opchar mutants for $ARGV[0] ($length residues)\n”; my $offset =
0; my $result = index($input, $char, $offset); while ($result != −1) { my $input0 = $input;
my $result1 = $result + 1; my $first = substr($input, 0, $result); my $point =
substr($input0, $result, 1) = $opchar; my $second = substr($input, $result1, $length);
my $opstring = $first.$point.$second; open OUTPUT, “> $char$result1$opchar−
$ARGV[0]” or die $!; print OUTPUT $opstring; close OUTPUT; print
“$char$result1$opchar−$ARGV[0] \n”; $offset = $result + 1; $result = index($input,
$char, $offset); }

### EXAMPLE 2

### High-Throughput Construction of Purified Plasmid Library Containing Encoded Mutant Genes

Due to codon degeneracy, many codons encode a single polypeptide. Species-specific codon tables such as http://www.kazusa.or.jp/codon/ (Yasukazu Nakamura, Kazusa) were used to construct primary insert structure for each wild-type construct. Secondary and tertiary structures were evaluated for negative expression affect and sequences were adjusted. Each wild-type construct was then cloned into an expression vector plasmid using standard technique. Point mutants were introduced by PCR-directed mutagenesis with a high-proof polymerase and two computationally generated unique primers per mutant. Compound mutants were created step-wise by point mutating each successively purified recombinant nucleic acid or by specialty multi point-mutant methods. Functional plasmid sequences were confirmed by automated sequencing and agarose electrophoresis.

### EXAMPLE 3

### High-Throughput Expression of Purified Plasmid Library and Purification of Expressed Polypeptide Library

Hosts were transformed with plasmid and incubated. Recombinant lysate was then processed and expressed polypeptides were roughly purified by non-column techniques. Filtrates were assayed by polyacrylamide electrophoresis followed by polypeptide staining.

### EXAMPLE 4

### High-Throughput Evaluation of Artificial Oxidoreductase Library for Activity and Other Industrial Properties

Polypeptides were assayed in 24-well plates for their respective reactions by spectrophotometry. Inclusion formation and solubilization were assayed by high-throughput methods.

1. An artificial mutant oxidoreductase polypeptide wherein the wild-type (unmutated) polypeptide is a human superoxide dismutase isoenzyme of superoxide dismutase 2 (hsod2) or superoxide dismutase 3 (hsod3) with a polypeptide sequence that is at least 70% identical to SEQ ID NO:1 or SEQ ID NO:2, said polypeptide having one or more mutations selected from the groups consisting of:
a. A substitution of Glu for Gln;
b. A substitution of Gln for Glu;
c. A substitution of Asp for Asn; and
d. A substitution of Asn for Asp.

2. An artificial mutant oxidoreductase polypeptide wherein the wild-type (unmutated) polypeptide is a human catalase (hcat) polypeptide with a polypeptide sequence that is at least 70% identical to SEQ ID NO:3, said polypeptide having one or more mutations selected from the groups consisting of:
a. A substitution of Glu for Gln;
b. A substitution of Gln for Glu;
c. A substitution of Asp for Asn; and
d. A substitution of Asn for Asp.

3. An artificial mutant oxidoreductase polypeptide wherein the wild-type (unmutated) polypeptide is a human glutathione peroxidase isoenzyme of glutathione peroxidase 1 isoform 1 (hgpx1-1) or glutathione peroxidase 1 isoform 2 (hgpx1-2) or glutathione peroxidase 2 (hgpx2) or glutathione peroxidase 3 (hgpx3) with a polypeptide sequence that is at least 70% identical to SEQ ID NO:4 or SEQ ID NO:5 or SEQ ID NO:6 or SEQ ID NO:7, said polypeptide having one or more mutations selected from the groups consisting of:
a. A substitution of Glu for Gln;
b. A substitution of Gln for Glu;
c. A substitution of Asp for Asn; and
d. A substitution of Asn for Asp.

4. A composition comprising the polypeptide of claim 1.

5. A composition comprising the polypeptide of claim 2.

6. A composition comprising the polypeptide of claim 3.
